(19) United States
(12) Patent Application Publication
(10) Pub. No.: US 2026/0306127 A1
(43) Pub. Date: Oct. 8, 2026

(54) Orbital Atherectomy Device with Exchangeable Burr System and Methods

(71) Applicant: Angiomed GmbH & Co. Medizintechnik KG, Karlsruhe (DE)

(72) Inventors: Sebastian Zickwolf, Bretten (DE); Lukas Oettinger, Linkenheim-Hochstetten (DE); Carolyn Benner, Karlsruhe (DE)

(21) Appl. No.: 19/172,972

(22) Filed: Apr. 8, 2025

(51) Int. Cl. A61B 17/3207; A61B 17/00; A61B 17/32; A61B 90/00

(52) U.S. Cl. CPC A61B 17/320758; A61B 17/00234; A61B 90/03; A61B 2017/00238; A61B 2017/00305; A61B 2017/00477; A61B 2017/00561; A61B 2017/320004; A61B 2090/035; A61B 2217/005

(57) ABSTRACT

An atherectomy system for intravascular occlusion ablation includes a handle and a catheter assembly extending longitudinally from the handle. The catheter assembly comprises a guide catheter defining a lumen, and a drive shaft that extends coaxially with the guide catheter from the handle to a distal tip. The drive shaft is configured to rotate about the longitudinal axis. The system further includes an ablation system featuring a one or more burrs that are releasably coupled to the drive shaft at either the distal tip or a location on the drive shaft proximal to the distal tip. This configuration facilitates the effective ablation of occlusions within blood vessels by allowing precise rotational control and positioning of the burr within the vascular system.

### BACKGROUND

Peripheral Arterial Disease (PAD) is a condition where the arteries that carry blood to the extremities (usually the legs) become narrowed or blocked, often due to atherosclerosis (plaque buildup). This can lead to reduced blood flow, causing symptoms like leg pain, cramping, or even more serious complications if untreated.

Atherectomy systems have been developed to treat PAD conditions and address the challenges of removing intravascular occlusions, such as plaque, from blood vessels. Traditional approaches have often involved the use of balloon angioplasty, where a balloon is inflated within the vessel to compress the plaque against the vessel walls. While this method can be effective in some cases, it may not adequately remove the plaque, and there is a risk of vessel damage or restenosis, where the vessel narrows again after treatment.

Another approach has been the use of laser atherectomy, which employs laser energy to vaporize or ablate the plaque. Laser atherectomy can be precise and effective in certain situations, particularly for softer plaque, but it may not be as effective for calcified or harder plaque. Additionally, the equipment required for laser atherectomy can be complex and costly, and the procedure may require specialized training.

Rotational atherectomy is another technique that has been employed, utilizing a rotating burr or other device to mechanically ablate the plaque. This method can be effective for calcified lesions and allows for the removal of plaque with minimal damage to the vessel wall. However, traditional rotational atherectomy systems may have limitations in terms of maneuverability and the ability to access certain areas within the vasculature, particularly in tortuous or narrow vessels. Often the burrs are permanently attached to the distal tip of the drive shaft of the device and as such require entirely different devices to provide different combinations of burrs depending on requirements of the procedure.

Disclosed herein are atherectomy systems and associated methods directed to address the foregoing.

### SUMMARY

In some aspects, the techniques described herein relate to an atherectomy system for ablating an occlusion disposed intravascularly including, a handle, and a catheter assembly extending along a longitudinal axis from the handle, the catheter assembly including, a guide catheter defining a lumen, a drive shaft extending coaxially with the guide catheter from the handle to a distal tip and configured to rotate about the longitudinal axis, and a first ablation system including a burr releasably coupled to the drive shaft at one of the distal tip or a point proximal of the distal tip.

In some aspects, the techniques described herein relate to an atherectomy system, wherein the burr includes an abrasive outer surface including one or more of diamond microchips or a textured surface.

In some aspects, the techniques described herein relate to an atherectomy system, wherein the burr further includes a non-abrasive surface.

In some aspects, the techniques described herein relate to an atherectomy system, wherein the burr defines a burr lumen extending along a central longitudinal axis and configured to receive a portion of the drive shaft therethrough, the burr defines a radial symmetry extending about the central longitudinal axis.

In some aspects, the techniques described herein relate to an atherectomy system, wherein the burr including the abrasive outer surface defines one of a cylindrical shape, a spherical shape, a three-dimensional “hourglass” shape, a conical shape, or a frusto-conical shape.

In some aspects, the techniques described herein relate to an atherectomy system, wherein the drive shaft includes a threaded outer surface, and the burr engages the drive shaft using a threaded engagement.

In some aspects, the techniques described herein relate to an atherectomy system, wherein the first ablation system further includes a proximal stop collar disposed proximally of the burr and a distal stop collar disposed distally of the burr.

In some aspects, the techniques described herein relate to an atherectomy system, wherein one or both of the proximal stop collar and the distal stop collar are coupled to the drive shaft using one of a threaded engagement, a press-fit engagement, a snap-fit engagement, a crimped engagement, adhesive, bonding, welding, or ultrasonic welding.

In some aspects, the techniques described herein relate to an atherectomy system, further includes a second ablation system coupled to a second location on the drive shaft, different from a first location of the first ablation system, the second location disposed proximally of the first location.

In some aspects, the techniques described herein relate to an atherectomy system, further including an aspiration system disposed in the handle, or in fluid communication with the handle, and configured to provide a vacuum to the lumen of the guide catheter to aspirate fragments of the occlusion.

In some aspects, the techniques described herein relate to an atherectomy system, further including a guidewire extending from the handle to a guidewire distal tip disposed distally of the distal tip of the drive shaft, the guidewire extending through a lumen defined by the drive shaft.

In some aspects, the techniques described herein relate to an atherectomy system, further including a guidewire extending from the handle to a guidewire distal tip disposed distally of the distal tip of the drive shaft, the guidewire extending through a lumen defined by the drive shaft.

In some aspects, the techniques described herein relate to an atherectomy system, wherein the burr includes a first half and a second half transitionable between an open configuration and a closed configuration, the burr configured to transitions to the closed configuration to engage a notch disposed in the drive shaft, the notch disposed proximally of the distal tip of the drive shaft.

In some aspects, the techniques described herein relate to an atherectomy system, wherein the notch defines a notch diameter that is less than an outermost diameter of the drive shaft, a diameter of the burr lumen being equal to, or slightly larger than the notch diameter and less than the outermost diameter of the drive shaft.

In some aspects, the techniques described herein relate to an atherectomy system, wherein the first half and the second half of the burr are hingedly coupled to each other along a first edge of the first half and a first edge of the second half by a hinge, the burr further including a latch disposed opposite the hinge and configured to releasably secure the burr in the closed configuration.

In some aspects, the techniques described herein relate to an atherectomy system, wherein the first half of the burr includes a first protrusion extending along a first edge, and a second protrusion extending along a second edge, the second half of the burr having a first groove extending longitudinally along a first edge and a second groove extending longitudinally along a second edge, the first half configured to slidably engage the second half along the longitudinal axis to secure the burr in a closed position.

In some aspects, the techniques described herein relate to an atherectomy system, wherein the notch include a facet configured to engage an abutment surface disposed within the burr lumen and configured to prevent rotation of the burr relative to the drive shaft when the burr is in the closed configuration.

In some aspects, the techniques described herein relate to a method of ablating an intravascular occlusion including, providing a drive shaft extending along a longitudinal axis to a distal tip, coupling one or more ablation burrs to the drive shaft at a position that is proximal of the distal tip, and rotating the drive shaft to rotate the one or more ablation burrs.

In some aspects, the techniques described herein relate to a method, wherein coupling the one or more ablation burrs to the drive shaft further includes, positioning a proximal stop collar at a first longitudinal position along the drive shaft, threadably engaging a burr of the one or more ablation burrs with the distal tip of the drive shaft, and rotating the burr relative to the drive shaft until the burr abuts against the proximal stop collar.

In some aspects, the techniques described herein relate to a method, wherein coupling the one or more ablation burrs to the drive shaft further includes transitioning a burr of the one or more ablation burrs to a closed configuration to engage a notch disposed in the drive shaft by engaging a first half of the burr with a second half of the burr, the notch extending through a burr lumen defined by burr in the closed configuration, a facet of the notch engaging an abutment surface of the burr to prevent rotation of the burr relative to the drive shaft.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of an atherectomy system generally including a handle and a catheter assembly, in accordance with embodiments disclosed herein.

FIG. 2 shows a longitudinal cross-section view of an ablation system of the atherectomy system for FIG. 1, in accordance with embodiments disclosed herein.

FIG. 3 shows a longitudinal cross-section view of an ablation system of the atherectomy system for FIG. 1, in accordance with embodiments disclosed herein.

FIGS. 4A-4B show perspective views of exemplary burrs, in accordance with embodiments disclosed herein.

FIG. 4C shows a lateral cross-section view of an exemplary burr, in accordance with embodiments disclosed herein.

FIGS. 4D-4E show perspective views of exemplary burrs, in accordance with embodiments disclosed herein.

FIG. 5A shows a longitudinal cross-section view of a drive shaft, in accordance with embodiments disclosed herein.

FIG. 5B shows a lateral cross-section view of the drive shaft of FIG. 5A, in accordance with embodiments disclosed herein.

FIGS. 6A-6D shows exemplary burrs of differing sizes engaged with a drive shaft, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter assembly or system disclosed herein includes a portion of the catheter assembly or system intended to be near or relatively nearer to a clinician when the catheter assembly or system is used on a patient. Likewise, a “proximal length” of, for example, the catheter assembly or system includes a length of the catheter assembly or system intended to be near or relatively nearer to the clinician when the catheter assembly or system is used on the patient. A “proximal end” of, for example, the catheter assembly or system includes an end of the catheter assembly or system intended to be near or relatively nearer to the clinician when the catheter assembly or system is used on the patient. The proximal portion, the proximal end portion, or the proximal length of the catheter assembly or system can include the proximal end of the catheter assembly or system; however, the proximal portion, the proximal end portion, or the proximal length of the catheter assembly or system need not include the proximal end of the catheter assembly or system. That is, unless context suggests otherwise, the proximal portion, the proximal end portion, or the proximal length of the catheter assembly or system is not necessarily a terminal portion or terminal length of the catheter assembly or system.

With respect to “distal,” a “distal portion” or a “distal end portion” of, for example, a catheter assembly or system disclosed herein includes a portion of the catheter assembly or system intended to be near or relatively nearer to a patient when the catheter assembly or system is used on a patient. Likewise, a “distal length” of, for example, the catheter assembly or system includes a length of the catheter assembly or system intended to be near or relatively nearer to the patient when the catheter assembly or system is used on the patient. A “distal end” of, for example, the catheter assembly or system includes an end of the catheter assembly or system intended to be near or relatively nearer to the patient when the catheter assembly or system is used on the patient. The distal portion, the distal end portion, or the distal length of the catheter assembly or system can include the distal end of the catheter assembly or system; however, the distal portion, the distal end portion, or the distal length of the catheter assembly or system need not include the distal end of the catheter assembly or system. That is, unless context suggests otherwise, the distal portion, the distal end portion, or the distal length of the catheter assembly or system is not necessarily a terminal portion or terminal length of the catheter assembly or system.

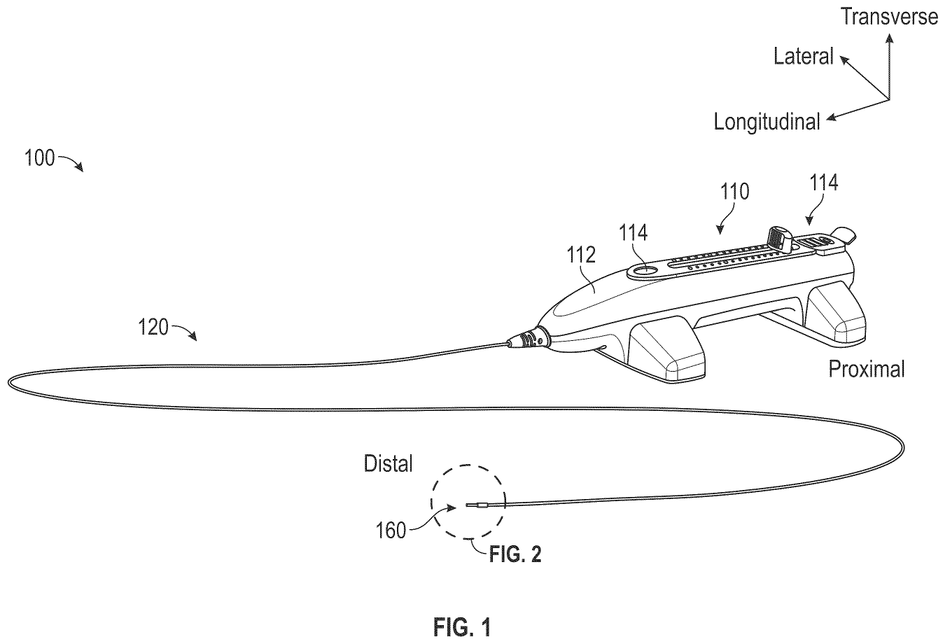

To assist in the description of embodiments described herein, as shown in FIG. 1, a longitudinal axis extends substantially parallel to an axial length of the catheter assembly. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIG. 1 shows a perspective view of an atherectomy system (“system”) 100 generally including a handle 110 and a catheter assembly 120. The handle 110 is disposed proximally and is configured to remain external to the patient. The catheter assembly 120 extends from a distal end of the handle 110 and generally includes one or more of a guide catheter 130, a drive shaft 140, and a guidewire 150, as described in more detail herein. A distal portion of the catheter assembly 120 is configured to be advanced through an insertion site into a vasculature of the patient.

In an embodiment, the handle 110 generally includes a body 112 extending longitudinally and configured to be grasped and manipulated by the clinician. The handle 110 further includes one or more controls 114 configured to perform various functions, including but not limited to advancing and/or retracting the catheter assembly 120 or one or more components thereof, rotating the catheter assembly 120 or one or more components thereof about a longitudinal axis, and providing a vacuum to the catheter assembly 120 or one or more components thereof. The controls 114 can include various buttons, switches, sliders, dials, touch sensitive interfaces, combinations thereof, or similar user interfaces to actuate one or more functions of the system.

In an embodiment, the handle 110 includes one or more of a power source, a motor, a biasing member such as a spring or the like, and/or one or more gear mechanisms, or combinations thereof, configured to provide rotational movement to the catheter assembly 120 or one or more components thereof. In an embodiment, the handle 110 includes an aspiration system including one or more of a vacuum pump, vacutainer, syringe, or similar mechanism configured to provide a vacuum to the catheter assembly 120 or one or more components thereof and aspire fragments of the occlusion from the intravascular target location.

In an embodiment, the handle can be coupled to additional systems configured to provide one or more of power, rotational movement or aspiration to the handle 110 that in turn can control providing power, rotational movement, or aspiration to the catheter assembly 120 or components thereof. For example, the handle 110 can be coupled to an external aspiration system, such as a medical vacuum system, or the like, and by actuation of the controls 114 can provide a vacuum to the catheter assembly 120. In the alternative, or in addition to, the handle 110 can be operably couple to one or more of a power source or a motor configured to provide rotational movement to the handle 110. By actuation of the controls 114, the handle 110 can then provide rotational movement to the catheter assembly 120. These and other configurations of handles 110 that provide one or more of rotational movement and/or vacuum to the catheter assembly 120 are contemplated to fall within the scope of the present invention.

The catheter assembly 120 extends from a distal end of the handle 110. The catheter assembly 120 generally includes one or more of a guide catheter 130, a drive shaft 140, and a guidewire 150, as described in more detail herein. Optionally the catheter assembly 120 can further include an aspiration catheter in addition to the guide catheter. In an embodiment, the guide catheter 130 can also function as an aspiration catheter. In an embodiment, one or more components of the catheter assembly 120 can be co-axially aligned. A distal portion of the catheter assembly 120 can include an ablation system 160.

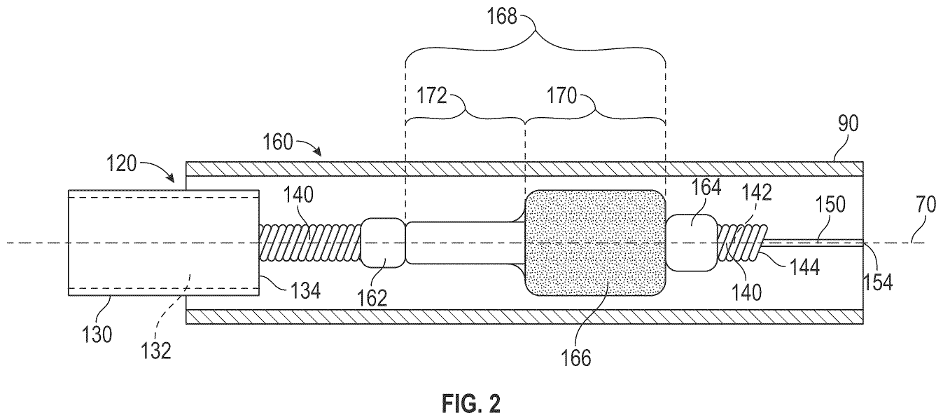

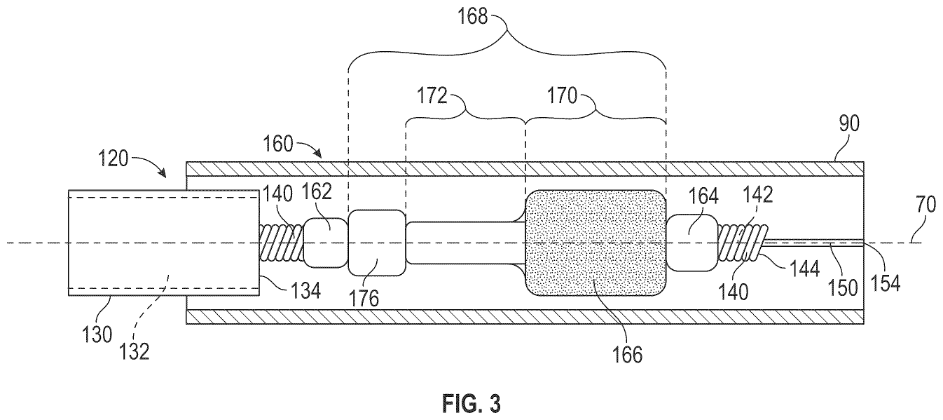

FIGS. 2-3 show embodiments of a catheter assembly 120 and ablation system 160 disposed within a vasculature 90 of a patient. The catheter assembly 120 includes a guide catheter 130 defining a lumen 132 and extending from the handle 110 at the proximal end, to an aperture 134 disposed at a distal tip. In an embodiment, a medical vacuum can be applied to a proximal end of the guide catheter lumen 132 to aspirate fluids or fragments of the occlusion dislodged by the ablation system 160. In an embodiment, the catheter assembly 120 can include a second catheter, different from the guide catheter 130 and extending parallel to, or coaxially with, the guide catheter 130 and configured to aspirate fluids or occlusion fragments, as described herein.

The catheter assembly 120 can further include a drive shaft 140 extending through the guide catheter lumen 132 and extending from the handle 110 at the proximal end to a distal tip 144. The handle 110 can be configured to advance or withdraw one or both of the guide catheter 130 and the drive shaft 140 axially along the longitudinally axis. The handle 110 can advance or withdraw the guide catheter 130 and the drive shaft 140 either concurrently or independently of each other. A distal tip 144 of the drive shaft 140 can extend distally of the aperture 134 at the distal end of the guide catheter 130.

In an embodiment, the drive shaft 140 is a solid, elongate wire. In an embodiment, the drive shaft 140 defines a lumen 142 through which a guidewire 150 extends. The drive shaft lumen 142 extends from the handle 110 at the proximal end to the distal tip 144 of the drive shaft 140. In an embodiment, the drive shaft is formed of a plastic, polymer, metal, alloy, composite, or combination thereof. In an embodiment, the drive shaft 140 is formed of a stainless steel, shape memory alloy, or similar suitable material. The drive shaft 140 can provide increased columnar strength to allow the drive shaft 140 to be urged axially through vasculature without kinking or collapsing, while still being flexible enough to negotiate tortuous vascular pathways. In an embodiment, the drive shaft 140 includes a threaded and/or a notched outer surface as described in more detail herein.

As shown in FIGS. 2-3, the catheter assembly 120 includes a guidewire 150 that extends through the guide catheter 130 and optionally through the drive shaft lumen 142. The guidewire 150 can be formed of a plastic, polymer, metal, alloy, composite, or combination thereof. In an embodiment, the guidewire 150 is formed of a stainless steel, shape memory alloy or similar suitable material. The guidewire 150 can provide increased columnar strength to allow the guidewire 150 to be urged axially through vasculature while still being flexible enough to negotiate tortuous vascular pathways. Once a distal tip 154 of the guidewire 150 is advanced to a target location within the vasculature 90, the guide catheter 130 and/or drive shaft 140 can be advanced over the guidewire 150 to the target location.

In an embodiment, the drive shaft 140 can include an ablation system 160 coupled to a distal portion thereof. The ablation system 160 can include a proximal stop collar 162, a distal stop collar 164, and a threaded burr 166. The proximal stop collar 162 can be coupled to the outer surface of the drive shaft 140 at a first longitudinal position. The distal stop collar 164 can be coupled to the outer surface of the drive shaft 140 at a second longitudinal position, distal of the first longitudinal position. A distance between the distal end of the proximal stop collar 162 and the proximal end of the distal stop collar 164 is termed a landing zone 168. The burr 166 can be disposed between the proximal stop collar 162 and the distal stop collar 164.

In an embodiment, the burr 166 can define a circular lateral cross-sectional shape and include an abrasive surface 170 disposed on an outermost surface thereof. In an embodiment, the burr 166 can define a radially symmetrical three-dimensional shape extending about a central longitudinal axis 70. Exemplary radially symmetrical three-dimensional shapes can include, but not limited to a cylindrical shape, a spherical shape, a three-dimensional “hourglass” shape, a conical shape, a frusto-conical shape, or the like. It will be appreciated that other three-dimensional shaped burrs are also contemplated. In an embodiment, the burr 166 can include one or more abrasive surfaces 170 disposed on an outer surface thereof. In an embodiment the burr 166 can vary in longitudinal length and diameter, as well as the number, longitudinal length, diameter, and position of the abrasive surface 170 disposed thereon.

In an embodiment, the abrasive surface 170 has a surface texture configured to ablate the occlusion as the burr 166 is rotated about the central longitudinal axis 70. In an embodiment, the burr 166 can further include a non-abrasive surface 172. The number, diameter, longitudinal position, longitudinal length of the abrasive surface 170 and/or the non-abrasive section of the burr 166 can vary depending on the different burr designs. In an embodiment, the abrasive surface 170 or the non-abrasive surface 172 can extend parallel to the longitudinal axis, or can extend at an angle relative to the longitudinal axis. In an embodiment, one or both of the abrasive surface 170 and the non-abrasive surface 172 can be convex or concave. In an embodiment, different abrasive surfaces 170 can include differing abrasive surfaces and/or different textures of abrasiveness. For example, the abrasive surface can include diamond microchips that range in size from 20 μm to 50 μm, or textured knurling engraved into a surface of the burr. As will be appreciated different configurations of burrs 166 can be provided. Further, a user can assemble or exchange different combinations of burrs 166 on the ablation system 160, as described herein.

In an embodiment, the longitudinal position of one or both of the proximal stop collar 162 and the distal stop collar 164 can be predetermined, i.e. fixed in place and unable to be moved by the clinician. In an embodiment, the longitudinal position of one or both of the proximal stop collar 162 and the distal stop collar 164 can be modified by the clinician. In an embodiment, one or more of the proximal stop collar 162, the distal stop collar 164, and the burr 166 can be coupled to the drive shaft 140 by a threaded engagement, press-fit engagement, snap-fit engagement, crimped, adhesive, bonding, welding, ultrasonic welding, combinations thereof, or the like.

For example, the proximal stop collar 162 can be welded to the drive shaft 140 and a predetermined location, the burr 166 can be threadably engaged with the drive shaft 140 from a distal tip 144 and rotated proximally until the burr 166 abuts against the proximal stop collar 162 preventing further rotation about the longitudinal axis and as such, prevents further proximal movement of the burr 166. The distal stop collar 164 can be threadably engaged with the drive shaft 140 from the distal tip 144 until the distal stop collar 164 abuts against the burr 166 locking the burr 166 in place. The ablation system 160 can be advanced through the vasculature to a target location. The handle 110 can rotate the drive shaft 140 about the longitudinal axis. The proximal stop collar 162 and the distal stop collar 164 secures the burr 166 to the drive shaft 140. As such, the drive shaft 140 causes the burr 166 to rotate about the central longitudinal axis 70. In an embodiment, the handle 110, and/or components thereof, can cause the burr 166 to rotate at high rotational speeds. Exemplary rotational speeds can be between 140,000 to 200,000 rotations per minute (“rpm”), however, greater or lesser rpm are also contemplated.

In an embodiment, the thread of the drive shaft 140 is either a right-handed thread or a left-handed thread. The rotation of the drive shaft 140 is either clockwise, or anti-clockwise. In an embodiment, the direction of the thread and the direction of the rotation can be configured to ensure the burr 166 abuts against the proximal stop collar 162 and mitigates movement of the burr 166 relative to the drive shaft 140 as the burr 166 is rotated at high rotational speeds. Advantageously, the ablation system 160 allows a clinician to modify the ablation system 160, to choose different types, shapes, or sizes of burr 166 as described herein. As such the clinician can modify the ablation system 160 in situ, before or during the procedure to suit the specific needs of the procedure.

As shown in FIG. 3, in an embodiment, the longitudinal length of the landing zone 168 is greater than the longitudinal length of the burr 166. As such, the clinician can further include a filler 176 slidably or threadably engaged with the drive shaft 140 and disposed between the proximal stop collar 162 and the distal stop collar 164. The filler 176 can be disposed either proximally or distally of the burr 166.

Advantageously, one or more ablation systems 160 can be coupled with the drive shaft 140 at different longitudinal locations. For example, a first ablation system 160, including a proximal stop collar 162, one or more burrs 166 and a distal stop collar 164, and optionally a filler 176 can be assembled at a first longitudinal location on the drive shaft 140. A second ablation system 160, including a proximal stop collar 162, one or more burrs 166 and a distal stop collar 164, and optionally a filler 176 can be assembled at a second longitudinal location on the drive shaft 140, e.g. proximally of the first longitudinal location. As will be appreciated, different combinations of ablation systems 160 can be assembled at different positions along a length of the drive shaft 140 by the clinician. The clinician can modify the configuration of the ablation system(s) 160 before or during the procedure as required.

In an exemplary method of use, an atherectomy system 100 is provided, as described herein. The system 100 includes one or more ablation systems 160 coupled to a distal portion of the drive shaft 140. Further, the clinician can modify the number and configuration of the one or more ablation systems 160, as described herein. In an embodiment, a clinician can threadably engage a burr 166 with a distal tip 144 of the drive shaft 140 and rotate the burr 166 to move the burr 166 proximally along the drive shaft 140 to a first longitudinal location along the drive shaft 140. As used herein, the first longitudinal location is disposed at a distal tip of the drive shaft 140 where the distal tip 144 is co-aligned with, or proximal to, a distal end of the ablation system 160. Further, as used herein, the first longitudinal location is disposed proximal of the distal tip of the drive shaft 140 where the distal tip 144 of the drive shaft 140 is distal to the distal end of the ablation system 160.

In an embodiment, a proximal stop collar 162 is crimped to the drive shaft 140 proximally of the burr 166. Optionally, the proximal stop collar 162 can be threadably engaged with the drive shaft 140 and includes, for example, a locking nut that mitigates rotation of the proximal stop collar 162 relative to the drive shaft 140 once positioned. As will be appreciated, the threaded proximal stop collar 162 can be positioned prior to positioning the burr 166. In an embodiment, the proximal stop collar 162 is welded, bonded, adhered, or otherwise fixed to the drive shaft 140 at a predetermined position. The clinician can then select the size, shape, or number of burrs 166 to be coupled with the drive shaft 140 and position the burr(s) 166 until they abut against the proximal stop collar 162. In an embodiment, a distal stop collar 164 is coupled to the drive shaft 140 distally of the burr 166 to prevent distal movement of the burr 166 relative to the drive shaft 140. Optionally, a filler 176 can be disposed between the proximal stop collar 162 and the distal stop collar 164 to align the burr(s) 166 as needed.

In an embodiment, a guidewire 150 is advanced to a target location within the vasculature. The guide catheter 130, drive shaft 140 and the one or more ablation systems 160 coupled thereto can be advanced over the guidewire 150 to the target location. The handle 110 can rotate the drive shaft 140 and apply a vacuum to the guide catheter lumen 132 to ablate and aspirate the occlusion.

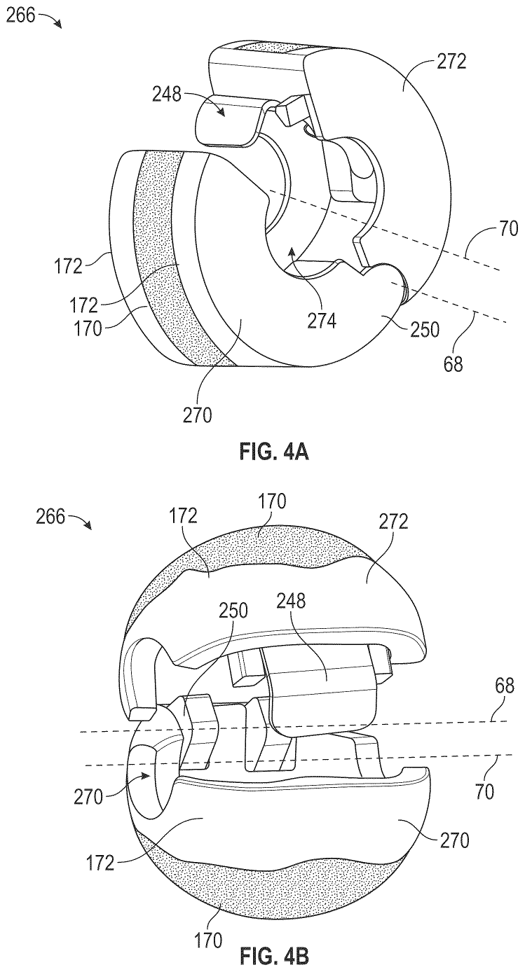

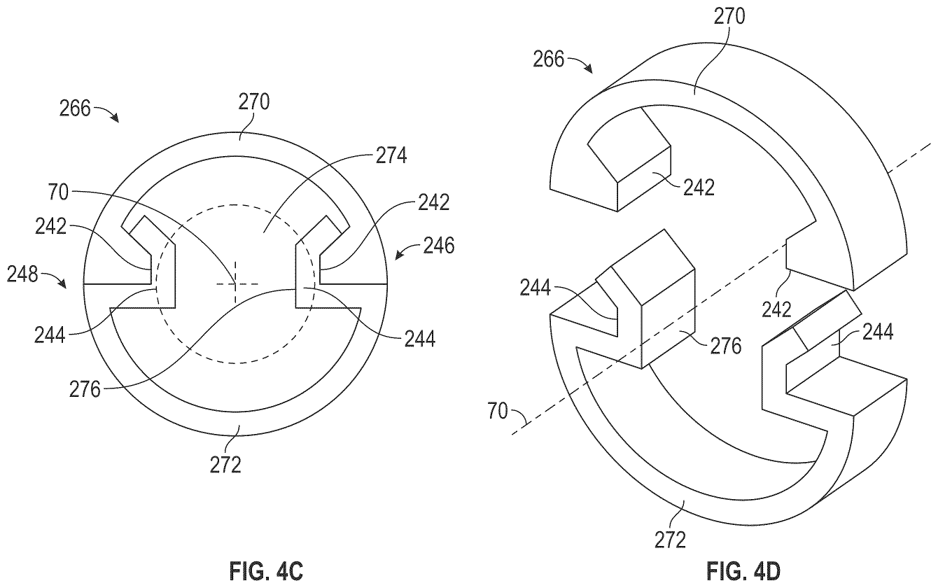

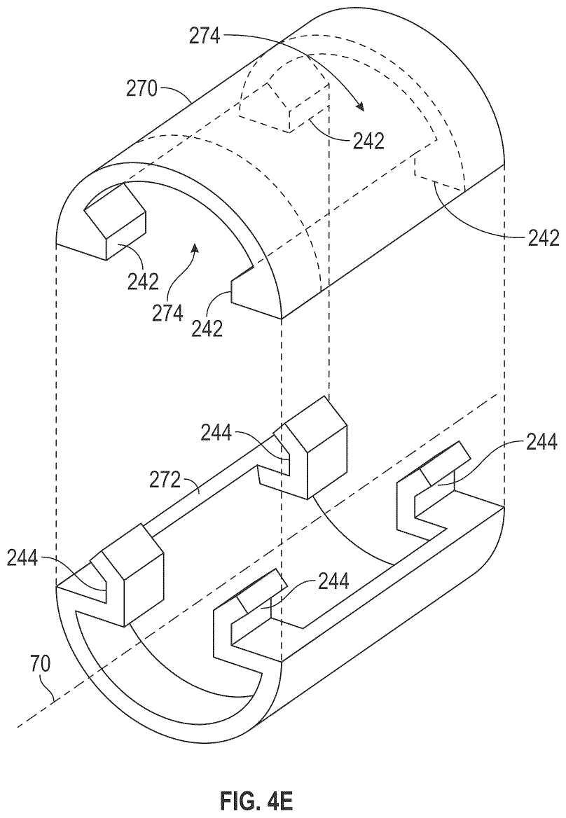

FIGS. 4A-6D show embodiments of an ablation system 260 to be used with the atherectomy system 100 and include one or more clip-on burrs 266. As shown in FIGS. 4A-4E, the clip-on burr 266 includes a first half 270 and a second half 272 that co-operate to define a burr 266 having radially symmetrical three-dimensional shape extending about a central longitudinal axis 70. Exemplary radially symmetrical three-dimensional shapes can include, but not limited to a cylindrical shape, a spherical shape, a three-dimensional “hourglass” shape, a conical shape, a frusto-conical shape, or the like, as described herein.

The first half 270 and a second half 272 of the burr 266 can transition between an open, or disassembled, configuration, and a closed, or assembled, configuration. In the assembled configuration, the first half 270 and a second half 272 of the burr 266 co-operated to define a three-dimensional shape having radial symmetry such as a cylinder (FIG. 4A) or a sphere (FIG. 4B), as described herein. However, it will be appreciated that other three-dimensional shapes have a radial symmetry about the central longitudinal axis 70 are also contemplated.

An outer surface of the burr 266 can include one or more of an abrasive surface 170 and a non-abrasive surface 172. The first half 270 and the second half 272 of the burr 266 can each include a notch. In the assembled configuration, the notches of the first half 270 and the second half 272 of the burr 266 co-operate to define a burr lumen 274 extending through the burr 266 along the central longitudinal axis 70. The burr lumen 274 can be configured to receive a portion of the drive shaft 240 therethrough.

In an embodiment, the first half 270 and the second half 272 of the burr 266 can be hingedly engaged to allow the first half 270 to pivot relative to the second half 272 of the burr 266 about a hinge axis 68. The hinge axis 68 extends parallel to the central longitudinal axis 70. The hinge 250 can couple a first edge of the first half 270 with a first edge of the second half 272. The second edge of the of the first half 270 and the second edge of the second half 272 can include a latching mechanism 248 to secure the first half 270 to the second half 272 in the closed configuration. The second edge of the of the first half 270 and the second edge of the second half 272 can be disposed opposite of the hinge 250 across the central longitudinal axis 70. Exemplary latching mechanisms 248 include a protrusion disposed on one of the first half 270 or the second half 272 and a detent disposed on the other of the first half 270 or the second half 272. The protrusion can engage the detent in an interference or snap-fit engagement to releasably secure burr 266 in a closed configuration. However, it will be appreciated that other latching mechanisms 248 configured to releasably secure the first half 270 to the second half 272 are also contemplated.

As best shown in FIG. 4C, in an embodiment, the inner surface of the burr lumen 274 includes an abutment surface 276 configured to engage a facet 278 disposed on the drive shaft 240. The facet 278 of the drive shaft 240 engages the abutment surface 276 and prevents rotation of the burr 266 relative to the drive shaft 240. As such, as the drive shaft 240 rotates, the drive shaft 240 can cause the burr 266 to rotate about the central longitudinal axis 70.

As shown in FIGS. 4C-4E, in an embodiment, the first half 270 and the second half 272 are coupled together using a first latching mechanism 248 and a second latching mechanism 246, the second latching mechanism 246 disposed opposite the first latching mechanism 248 across a central longitudinal axis 70. FIG. 4C shows a lateral cross-section view of the burr 266 in the assembled, or closed, configuration, with each of the first latching mechanism 248 and the second latching mechanism 246 engaged. FIG. 4D shows a perspective, exploded view of the burr 266 in the disassembled, or open, configuration.

In an embodiment, one or both of the first latching mechanism 248 and the second latching mechanism 246 include a snap-fit engagement. A protrusion 242 of the first latching mechanism 248 of the first half can engage a detent 244 of the first latching mechanism 248 of the second half 272. The first half 270 can be pivoted and urged down on to the second half 272 causing the protrusion 242 of the second latching mechanism 246 to flex radially outwards before engaging the detent 244 of the second latching mechanism 246.

In an embodiment, the first half 270 slidable engages the second half 272 by sliding the protrusions 242 of the first half 270 into the detents 244 of the second half 272 along the longitudinal axis. These and other latching mechanisms configured to releasably engage a first half 270 with a second half 272 and secure the burr 266 to the drive shaft 240 are also contemplated. For example, as shown in FIG. 4E, a burr 266 can include an elongate cylindrical shape extending along the longitudinal axis. The burr 266 includes two or more latching mechanisms, as described herein, to secure the first half 270 to the second half 272.

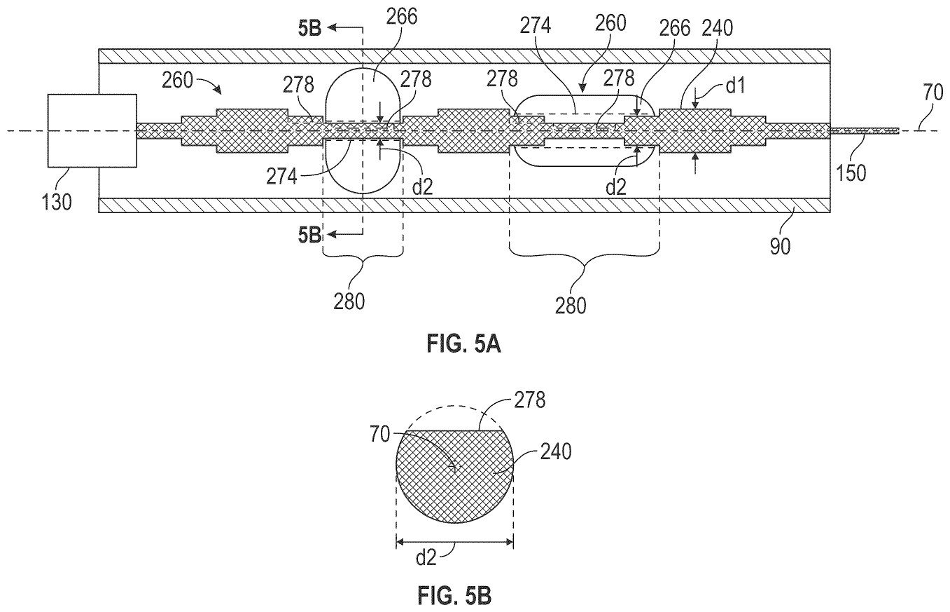

As shown in FIGS. 5A-5B, in an embodiment, the drive shaft 240 can include one or more notches 280 disposed along the longitudinal length thereof. A longitudinal length of the notch can be configured to be equal to, or slightly longer than, a longitudinal length of the burr 266. Further a diameter drive shaft 240 within the notch 280, e.g. a notch diameter (d2) can be equal to, or slightly less than, a diameter of the burr lumen 274. As such, the clip-on burr 266 can be clipped to the notch 280, the diameter of the drive shaft in the notch 280 can fit tightly within the burr lumen 274. To note, the outer diameter (d1) of the drive shaft 240 is greater than the diameter of the burr lumen 274. As such, the proximal and distal ends of the notch 280 can abut against the ends of the burr 266 preventing the burr 266 from sliding longitudinally relative to the drive shaft 240.

In an embodiment, the drive shaft 240 can include different numbers, locations, longitudinal lengths, notch diameters of notches 280 in order to accommodate different size, shapes, and burr lumen diameters of different burrs 266.

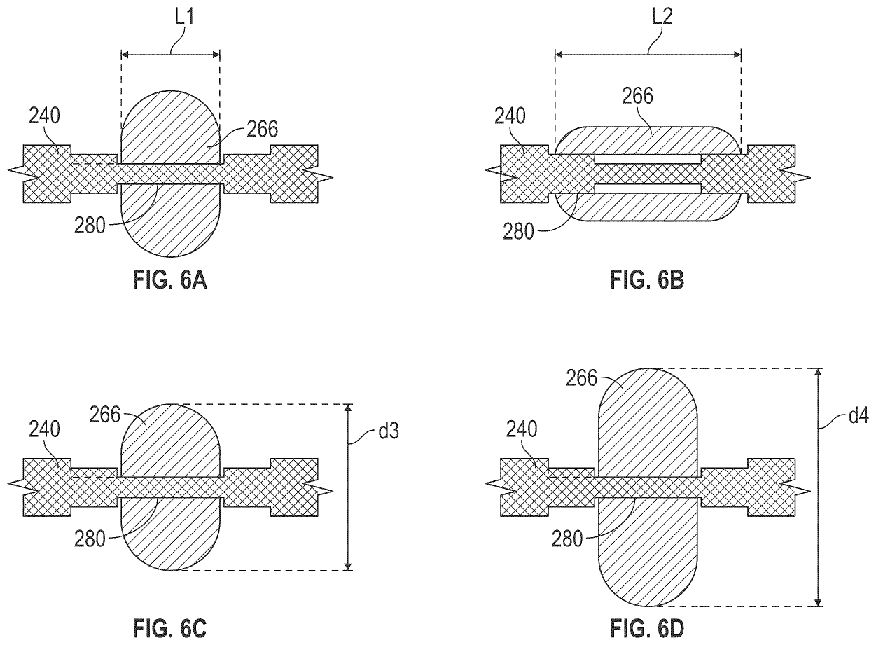

FIGS. 6A-6D show exemplary embodiments of clip-on burrs 266 having varying longitudinal lengths (e.g. L1, L2), diameters (e.g. d3, d4), and shapes and coupled to notches 280 on a drive shaft 240 and having differing longitudinal lengths and diameters. Advantageously, the clinician can modify the number, location and type of burrs 266 coupled to the drive shaft by clipping or unclipping different burrs 266 to/from the drive shaft. As such the clinician can modify the configuration of the ablation system 260 prior to, or during a procedure.

In an exemplary method of use, an atherectomy system 100 is provided as described herein. The system 100 includes one or more ablation systems 260 coupled to a distal portion of the drive shaft 240. Further, the clinician can modify number and configuration of the one or more ablation systems 260. In an embodiment, a clinician can engage a burr 266 with a notch 280 disposed on the drive shaft 240.

In an embodiment, a burr 266 transitions to a closed, or assembled, configuration encircling a notch 280 of the drive shaft 240. A first half 270 of the burr 266 can be hingedly coupled to a second half 272 of the burr 266 to facilitate transitioning between the open and closed configurations. In an embodiment, the burr 266 can include one or more latching mechanisms, e.g. a first latching mechanism 248 and a second latching mechanism 246 to secure the burr 266 in the assembled configuration.

In the assembled configuration the burr 266 is secured to the drive shaft 240 by receiving a portion of the drive shaft, e.g. a notch 280, through the burr lumen 274. A notch diameter (d2) is equal to, or slightly less than, the burr lumen 274 diameter. Further, a longitudinal length of the notch 280 is equal to, or slightly longer than, a longitudinal length of the burr 266, (e.g., L1 or L2). As such, the notch 280 secures the burr 266 at the location and mitigates longitudinal movement of the burr 266 relative to the drive shaft 240. In an embodiment, the notch includes a facet 278 that engages an abutment surface 276 disposed within the lumen 274 of the burr 266. The facet 278 engages the abutment surface 276 and prevents rotation of the burr 266 relative to the drive shaft 240.

In an embodiment, a guidewire 150 is advanced to a target location within the vasculature. The guide catheter 130, drive shaft 240 and the one or more ablation systems 260 coupled thereto can be advanced over the guidewire 150 to the target location. The handle 110 can rotate the drive shaft 240 to rotate the burr 266. The abrasive surface 170 can ablate the occlusion as the burr 266 rotates. The handle 110 applies a vacuum to the guide catheter lumen 132 to aspirate the occlusion.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An atherectomy system for ablating an occlusion disposed intravascularly, comprising:
a handle; and
a catheter assembly extending along a longitudinal axis from the handle, the catheter assembly comprising:
a guide catheter defining a lumen;
a drive shaft extending coaxially with the guide catheter from the handle to a distal tip and configured to rotate about the longitudinal axis; and
a first ablation system including a burr releasably coupled to the drive shaft at one of the distal tip or a point proximal of the distal tip.

2. The atherectomy system according to claim 1, wherein the burr includes an abrasive outer surface including one or more of diamond microchips or a textured surface.

3. The atherectomy system according to claim 2, wherein the burr further includes a non-abrasive surface.

4. The atherectomy system according to claim 2, wherein the burr defines a burr lumen extending along a central longitudinal axis and configured to receive a portion of the drive shaft therethrough, the burr defines a radial symmetry extending about the central longitudinal axis.

5. The atherectomy system according to claim 4, wherein the burr including the abrasive outer surface defines one of a cylindrical shape, a spherical shape, a three-dimensional “hourglass” shape, a conical shape, or a frusto-conical shape.

6. The atherectomy system according to claim 1, wherein the drive shaft includes a threaded outer surface and the burr engages the drive shaft using a threaded engagement.

7. The atherectomy system according to claim 1, wherein the first ablation system further includes a proximal stop collar disposed proximally of the burr and a distal stop collar disposed distally of the burr.

8. The atherectomy system according to claim 7, wherein one or both of the proximal stop collar and the distal stop collar are coupled to the drive shaft using one of a threaded engagement, a press-fit engagement, a snap-fit engagement, a crimped engagement, adhesive, bonding, welding, or ultrasonic welding.

9. The atherectomy system according to claim 1, further includes a second ablation system coupled to a second location on the drive shaft, different from a first location of the first ablation system, the second location disposed proximally of the first location.

10. The atherectomy system according to claim 1, further including an aspiration system disposed in the handle, or in fluid communication with the handle, and configured to provide a vacuum to the lumen of the guide catheter to aspirate fragments of the occlusion.

11. The atherectomy system according to claim 1, further including a guidewire extending from the handle to a guidewire distal tip disposed distally of the distal tip of the drive shaft, the guidewire extending through a lumen defined by the drive shaft.

12. The atherectomy system according to claim 1, further including a guidewire extending from the handle to a guidewire distal tip disposed distally of the distal tip of the drive shaft, the guidewire extending through a lumen defined by the drive shaft.

13. The atherectomy system according to claim 4, wherein the burr includes a first half and a second half transitionable between an open configuration and a closed configuration, the burr configured to transitions to the closed configuration to engage a notch disposed in the drive shaft, the notch disposed proximally of the distal tip of the drive shaft.

14. The atherectomy system according to claim 13, wherein the notch defines a notch diameter that is less than an outermost diameter of the drive shaft, a diameter of the burr lumen being equal to, or slightly larger than the notch diameter and less than the outermost diameter of the drive shaft.

15. The atherectomy system according to claim 13, wherein the first half and the second half of the burr are hingedly coupled to each other along a first edge of the first half and a first edge of the second half by a hinge, the burr further including a latch disposed opposite the hinge and configured to releasably secure the burr in the closed configuration.

16. The atherectomy system according to claim 13, wherein the first half of the burr includes a first protrusion extending along a first edge, and a second protrusion extending along a second edge, the second half of the burr having a first groove extending longitudinally along a first edge and a second groove extending longitudinally along a second edge, the first half configured to slidably engage the second half along the longitudinal axis to secure the burr in a closed position.

17. The atherectomy system according to claim 13, wherein the notch include a facet configured to engage an abutment surface disposed within the burr lumen and configured to prevent rotation of the burr relative to the drive shaft when the burr is in the closed configuration.

18. A method of ablating an intravascular occlusion, comprising:
providing a drive shaft extending along a longitudinal axis to a distal tip;
coupling one or more ablation burrs to the drive shaft at a position that is proximal of the distal tip; and
rotating the drive shaft to rotate the one or more ablation burrs.

19. The method according to claim 18, wherein coupling the one or more ablation burrs to the drive shaft further includes:
positioning a proximal stop collar at a first longitudinal position along the drive shaft;
threadably engaging a burr of the one or more ablation burrs with the distal tip of the drive shaft; and
rotating the burr relative to the drive shaft until the burr abuts against the proximal stop collar.

20. The method according to claim 18, wherein coupling the one or more ablation burrs to the drive shaft further includes transitioning a burr of the one or more ablation burrs to a closed configuration to engage a notch disposed in the drive shaft by engaging a first half of the burr with a second half of the burr, the notch extending through a burr lumen defined by burr in the closed configuration, a facet of the notch engaging an abutment surface of the burr to prevent rotation of the burr relative to the drive shaft.
